(19) United States
(12) Patent Application Publication
(10) Pub. No.: US 2020/0219596 A1
(43) Pub. Date: Jul. 9, 2020

(54) SYSTEMS AND METHODS FOR MANAGING PROTECTED INFORMATION ACCESS AND CONSENT TO ACCESS

(71) Applicant: Particle Health, Inc., Brooklyn, NY (US)

(72) Inventors: Troy BANNISTER, Brooklyn, NY (US); Dan Horbatt, Jersey City, NJ (US)

(73) Assignee: Particle Health, Inc., Brooklyn, NY (US)

(21) Appl. No.: 16/736,294

(22) Filed: Jan. 7, 2020

Related U.S. Application Data
(60) Provisional application No. 62/789,244, filed on Jan. 7, 2019.

(51) Int. Cl. G16H 10/60; G06Q 50/22; G16H 15/00; G06F 21/32; G06F 21/62

(52) U.S. Cl. CPC G16H 10/60; G06Q 50/22; G06F 21/6245; G06F 21/32; G16H 15/00

(57) ABSTRACT

Methods and systems are provided for retrieving consent from a data owner for disbursement of information associated with the data owner. A data requester provides a request for information (ROI) identifying information sought and the associated data owner. The data owner receives a consent request and provides consent to some or all of the requested information being provided to the data requester. The requested information is retrieved from a corresponding data store and provided to the data requester based on the provided consent.

### CROSS-REFERENCE TO RELATED APPLICATION

This application is related to and claims priority under 35 U.S.C. § 119(e) from U.S. Patent Application No. 62/789,244, filed Jan. 7, 2019 entitled “System and Method to Manage Consent,” the entire contents of which is incorporated herein by reference for all purposes.

### TECHNICAL FIELD

Aspects of the present disclosure relate to protected information storage and retrieval and, more specifically, to consent management for retrieving protected information by a requester.

### BACKGROUND

Various information is protected by regulation and requires or strongly benefits from documented consent to access said information. For example, medical records typically require patient consent retrieve, access, and distribute to requesting parties, such as physicians, insurers, etc. Further, medical records, and other protected information, are typically fragmented across multiple healthcare providers, storage services, and other entities which need to access and update said records. As a result, it is generally a tedious and labor intensive process for a healthcare provider to gain access to a patient medical record. Consent must be received from the patient and communicated to the various entities storing the sought medical records and various other forms and process are typically involved in order to provide an audit trail for all the involved parties. The consent is generally provided by the patient each and every time a record is to be transferred and very often requires the patient to engage in a large amount of effort and navigate various bureaucratic processes to simply provide consent. As a result, an immense amount of time, energy, and resources is spent by all parties in transferring patient medicals records between entities.

It is with these observations in mind, among others, that various aspects of the present disclosure were conceived.

### SUMMARY

Embodiments of the invention concern systems and methods for providing information associated with a data owner to a data requester based on consent of the data owner.

A computer-implemented method for delivering consented to information includes receiving, from a data requester, a request for consented to information associated with a data owner, retrieving, from the data owner, consent to deliver the consented to information to the data requester, determining, based on the request and the data owner, a data store from which to retrieve the consented to information, retrieving, from the determined data store, the consented to information, and delivering the consented to information to the data requester.

In one embodiment of the computer-implemented method, the method further includes validating the retrieved consent, wherein validation is based on one or more of biometric information, a password, a code, or a key.

In one embodiment of the computer-implemented method, the method further includes modifying the retrieved consented to information based on the retrieved consent, and wherein the modified retrieved consented to information is delivered to the data requester.

In one embodiment of the computer-implemented method, the method further includes generating a consent profile for the data owner, and wherein retrieving the consent from the data owner includes accessing the generated consent profile.

In one embodiment of the computer-implemented method, the consent is retrieved from the data owner through a mobile application deployed to a mobile device.

In one embodiment of the computer-implemented method, retrieving the consent to information from the determined data store further includes generating a second request based on the received request, the second request complying with one of an application programming interface (API) or a protocol corresponding to the determined data store.

In one embodiment of the computer-implemented method, one or more of the data requester is a health provider, the data owner is a patient, or the requested consented to information associated with the data owner is part of a medical record.

A system for delivering consented to information includes one or more processors, and a memory including instructions to receive, from a data requester, a request for consented to information associated with a data owner, retrieve, from the data owner, consent to deliver the consented to information to the data requester, determine, based on the request and the data owner, a data store from which to retrieve the consented to information, retrieve, from the determined data store, the consented to information, and deliver the consented to information to the data requester.

In one embodiment of the system, the memory further includes instructions to validate the retrieved consent, wherein validation is based on one or more of biometric information, a password, a code, or a key.

In one embodiment of the system, the memory further includes instructions to modify the retrieved consented to information based on the retrieved consent, and wherein the modified retrieved consented to information is delivered to the data requester.

In one embodiment of the system, the memory further includes instructions to generate a consent profile for the data owner, and wherein retrieving the consent from the data owner includes accessing the generated consent profile.

In one embodiment of the system, the consent is retrieved from the data owner through a mobile application deployed to a mobile device.

In one embodiment of the system, retrieving the consent to information from the determined data store further includes generating a second request based on the received request, the second request complying with one of an application programming interface (API) or a protocol corresponding to the determined data store.

In one embodiment of the system, one or more of the data requester is a health provider, the data owner is a patient, or the requested consented to information associated with the data owner is part of a medical record.

A non-transitory computer readable medium stores instructions that, when executed by one or more processors, cause the one or more processors to receive, from a data requester, a request for consented to information associated with a data owner, retrieve, from the data owner, consent to deliver the consented to information to the data requester, determine, based on the request and the data owner, a data store from which to retrieve the consented to information, retrieve, from the determined data store, the consented to information, and deliver the consented to information to the data requester.

In one embodiment of the non-transitory computer readable medium, instructions are further stored to validate the retrieved consent, wherein validation is based on one or more of biometric information, a password, a code, or a key.

In one embodiment of the non-transitory computer readable medium, instructions are further stored to modify the retrieved consented to information based on the retrieved consent, and wherein the modified retrieved consented to information is delivered to the data requester.

In one embodiment of the non-transitory computer readable medium, instructions are further stored to generate a consent profile for the data owner, wherein retrieving the consent from the data owner includes accessing the generated consent profile.

In one embodiment of the non-transitory computer readable medium, the consent is retrieved from the data owner through a mobile application deployed to a mobile device.

In one embodiment of the non-transitory computer readable medium, retrieving the consent to information from the determined data store further includes generating a second request based on the received request, the second request complying with one of an application programming interface (API) or a protocol corresponding to the determined data store.

### BRIEF DESCRIPTION OF THE DRAWINGS

FIG. 1 is a systems diagram illustrating an operating environment for a health consent management application, in accordance with various embodiments of the subject technology;

FIG. 2 is a sequence diagram illustrating an example operation pattern of a health consent management application, in accordance with various embodiments of the subject technology;

FIG. 3 is a flowchart depicting an example method for managing patient consent and medical records retrieval, in accordance with various embodiments of the subject technology; and

FIG. 4 is a systems diagram of an example computing system that may implement various systems and methods as disclosed herein, in accordance with various embodiments of the subject technology.

### DETAILED DESCRIPTION

For purposes of clarity and understanding, the disclosure uses certain terms in an explicitly defined manner. However, it is understood that said terms are defined for purposes of explanation only and not to be taken as unduly limiting. For example, ‘data seekers’ are entities wishing to acquire medical records of a patient for the purpose of facilitating some business operation (e.g., a life insurance underwriter, a healthcare provider, etc.).

A consent management application, including various systems and methods, is disclosed for providing regulatory compliant medical record retrieval and display for a data seeker. Health information exchanges (HIEs) are integrated into the systems and methods to support seamless retrieval of medical records. In some examples, the HIEs are incentivized to support the consent management application, which represents an additional monetization stream that may be layered atop provisioning of medical records.

Requests for information (ROIs), and specifically requests for patient medical records, can be processed in minutes or less, rather than in the months it may often take in typical medical records systems. Revenue streams for all downstream parties (e.g., consent management services, HIEs, originating institutions, etc.) may also be tracked. Respective patients are provided with insight into the processing of respective ROIs and can achieve intuitive and cohesive sharing control over their own medical data.

The consent management application integrates a multitude of application programming interfaces (APIs) to access and interface with different databases storing and managing patient medical records. As a result, data can be pulled using protected health information (PHI) over a unified query-based protocol. Thus, each system storing relevant medical records data does not need to be redundantly queried individually by a patient and/or data seeker.

The consent management application uses the API integrations to automatically query each relevant database, or data silo, for the respective records in accordance to each of the policies and procedures of each respective data silo. In effect, a single request provided to the consent management application can be used to search and pull multiple datasets from multiple databases for a single individual.

In general, ROIs are drive the processing flow of the consent management application. The ROIs are modeled as a workflow that is started by a data seeker filling out a request. The filled out request is then used to query integrated systems, gain consent of the party for which information is being requested (e.g., the “data owner”), and provide a cohesive report of all the data that has been retrieved. The request may act as the central point of interaction with this data, for both the requesting party as well as the data owner, allowing them to edit and/or cancel the request as needed.

In some examples, the consent management application includes, or is part of, a health information management system (HIMS). When requesting health information for a data owner, the requesting party is directed to the consent management application through a HIMS website or API plugin (e.g., where the consent management application is integrated into a third party HIMS, etc.). The requesting party fills out a ROI form accessible through the consent management application. In some examples, the ROI form includes automations to assist in completing, or to provide validation of information entered into, the ROI form.

The request is then validated and the data owner is automatically contacted to verify and consent to the request. Once consent is obtained, the requesting party is notified they have consent to view the data, and may pay to release the information. In some examples, a fee may be paid for information on a per silo basis. In some examples, the requesting party may have a subscription or similar membership granting bulk access. In some examples, information may be obtained through a combination of bulk and per silo bases.

Nevertheless, the requesting party can download and/or view all raw data consented to by the data owner. In some examples, the requesting party may generate a report through the consent management application providing the various accessed medical records in a unified and aggregated format.

Once the data owner revokes the request, or the request expires, the consent management application may archive the request for future reference. Where the request is archived, respective health data is removed from the system and made unavailable without another ROI and respective consent from the data owner. In addition, the consent management application supports further workflows, such as, for example and without limitation, registrations, edit cycles, internal errors from integrations, etc.

As discussed above, the consent management application includes integrations for interfacing with various data silos from which medical records may be retrieved. In particular, various HIEs can be integrated into the consent management application via conformant API calls and other querying protocols. The integrations comply with public and respective private industry standards, such as, for example and without imputing limitation, fast health care interoperability resources (FHIR) standard, InterSystems® IRIS™, or the like.

The API and query calls pass in PHI included as part of the ROI to query against the a Master Patient Index of a respective data silo and retrieve all records the system contains on any resulting matches. Where supported, justification and/or explanation of the query (e.g., new patient intake, insurance underwriting, record update, etc.) is also passed along via API and/or query calls.

In particular, patient authorization can be used to search data silos for patient data using PHI associated with the authorization. As a result, data for an individual can be pulled from any number of data silos including patient identifying information. In some examples, such as for data silos which do not integrate PHI into a usable query protocol, the PHI can be used to track back to a reference database storing appropriate corresponding information.

Raw data returned from the integrations with health data providers may be made available to both the respective patient and/or respective requesting party. Further, a comprehensive report can be generated that combines the various sources of data into one cohesive, time-series report. Additionally, the comprehensive report may be further enhanced by applying additional analytics to the raw data to create flags and scores for relevant use cases.

The comprehensive report is available in several different fashions. In one example, an interactive chart can be provided via the consent management application through a graphical user interface (GUI) or the like. The interactive chart may allow for searching, filtering, and interactive graphs to navigate and pay for access to certain reports and/or sources of data (e.g., records from certain data silos, etc.). In some examples, where a requesting system integrating the consent management application (e.g., via API integrations, etc.) supports it, data can be loaded directly into the requesting system. In some examples, a downloadable PDF can be made available for offline viewing of the report, etc.

ROIs can be secured by a strict role based access control policy. Each ROI is associated with, and visible to, a respective data owner as well as the requesting party. In effect, patients have the opportunity to additionally share the requested information out with other entities (e.g., a referring doctor, etc.). In some examples, data seekers may create roles for different teams to manage multiple requests in a secure manner. For example, a large organization seeking health information for multiple patients may assign various teams to each patient using a role-based system and so minimize risk of information becoming accidentally mixed between records or the like. In some examples, any change or information that is requested outside of normal operating procedures requires intervention by a specialized team associated with the consent management application. For example, an audit log cataloging who accessed which data and when may be requested from the specialized team and provided to a data seeker, such as for compliance investigations and the like.

A full audit trail can be logged cataloging all requests, regardless of whether a ROI successfully returned patient records, etc. All request transitions and raw data viewings can be associated with an immutable log indicating a resource being operated on (e.g., a terminal, or computer, identifier, etc.), an operating party (e.g., a requester or data seeker login, etc.), when the operation took place (e.g., a timestamp, etc.), and the result of the operation (e.g., a null result, patient data identifier, actual report generated, etc.). The logged information can be made available to a respective data owner and the requesting party so they are aware of how the data is being used.

Typical processes for requesting medical records lack various security and trust capabilities. The consent management application may integrate various points of trust and security for both data requesters and data owners into the process for retrieving information. In one example, the consent management application requires an account to make a request for data. Accounts may undergo vetting and validation prior to being authorized to make ROIs, ensuring that requests are from organizations having an actual business need to access medical records information. Additionally, as an account continues to use the consent management application, a reputation of trust and record of requests and information usage associated with the account is created and grown.

In some examples, when validating a request for records, the associated data owner is queried for information that is present in the requested records in order to ensure that they are who they say they are. For example, and without imputing limitation, the validation query may be a simple identification of the data owner's primary care physician or may be a more complex validation procedure such as a personal identification number, social security number, or the like.

The consent management application further reduces and/or avoids redundant completion of multiple authorization forms by data owners. In particular, a common and universal consent module is leveraged which satisfies regulatory requirements and serves as a single request point for multiple data silos storing medical records. In some examples, the consent module may further include additional explanatory text and granular permissions for data owners to provide informed and narrow consent for information disbursement. Additionally, the consent management application allows for data seekers to upload and provide existent consents and authorizations where respective signatures have already retained earlier.

Upon onboarding, users (e.g., data owners) are able to build up a consent profile for future use. A consent profile is a mechanism that allows for automatic approval and/or declination of a request from a data seeker to gain access to respective health information. In some examples, the user has full control over building a set of rules governing the consent and/or consent profiles. For example, and without imputing limitation, time limits for which consent may be in force can be specified, subsets of use cases (e.g. nonprofit research, insurance, etc.) can be linked to certain consents, and/or specific organizations can be listed that the user would like to automatically allow or deny requests from without having their need to physically interact with the platform.

Additionally, in some examples, further filters can be applied to health information produced for data seekers. For example, certain aspects of medical records may be automatically filtered out of reports or the like (e.g., in compliance with regulations covering substance abuse, sexual and mental health history, etc.).

In some examples, the consent management application may provide rich analytics regarding ROIs sent to various integrated data silos (e.g., HIEs, etc.). Administrators from the integrated institutions may use the rich analytics, for example, to gain insight into the number of requests targeting respective organizations at various phases in the process of transmitting health information responsive to data seeker requests. As a result, increased transparency into the ROI process may be provided to institutional parties to the process (e.g., HIEs, etc.), as well as reporting for payment remuneration purposes and the like when it comes to divvying revenue from requests.

Analytics may be available as a fine grained list of all requests targeting data held by a respective institution. Additionally, GUIs supporting the analytics may include, for example and without imputing limitation, sorting, filtering, search capabilities, etc. In some examples, coarse time-series charting supporting sorting and filtering capabilities is also provided. Further, time period reporting, in the form of invoices, may also be available.

In effect, the consent management application may provide a one-stop shop for authentication and authorization of data seekers and/or data owners. Data owners need only to provide the consent management application with appropriate information and preferences, and the consent management application may authorize multiple requesters to view their information without requiring tedious and duplicative paperwork and form-filling. As a result, the burden on patients is significantly reduced by providing a trusted central authority that can verify data requests to multiple data providers.

Data owners do not have to repeatedly authenticate themselves, which reduces cost and allows for easy connections and integrations with new partners. In addition, complexity around authentication and authorization is reduced and so industry best practices around security can be enforced by reducing points of failure. Data providers do not need to invest in bespoke authentication and authorization methods and procedures, reducing training and development costs. Data requestors also need not learn a new system for each provider. As a result, an increase in speed and efficiency in health providers serving patients is realized and trust across all aspects of the patient health information pipeline is increased.

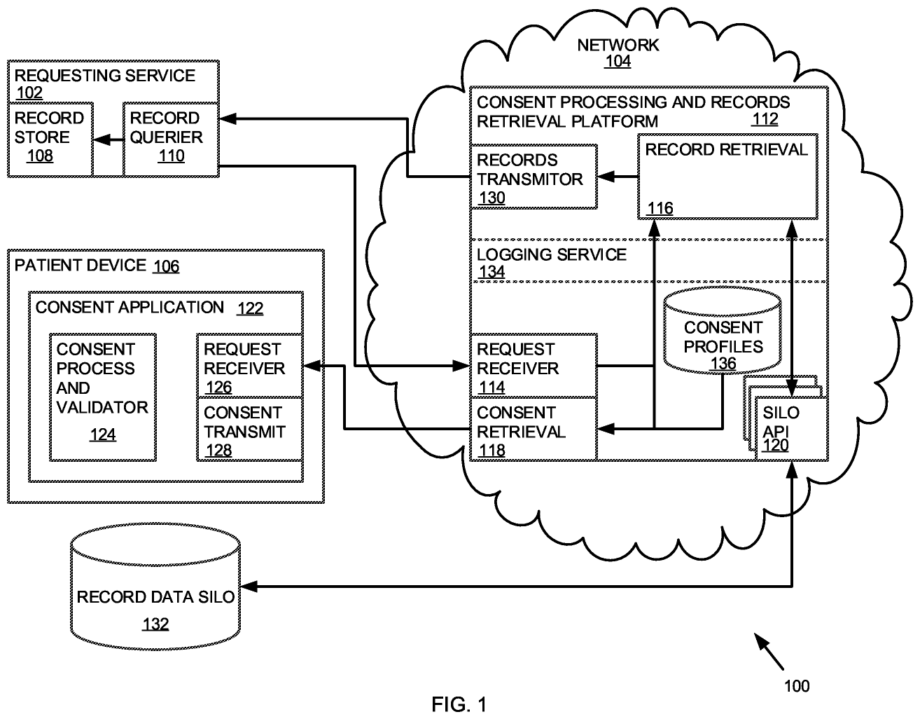

FIG. 1 depicts an operating environment 100 in which a consent management application may manage requests for information, such as health information, medical records, personal information, and other consent managed information records, from a data requester (e.g., a clinic, physician, insurer, etc.). The consent management application acquires appropriate consent from a corresponding data owner (e.g., patient, client, etc.) and performs consented to disbursements of the requested information.

In general, a requesting service 102 is installed on a computing device, system, or network of the data requester. In some examples, requesting service 102 is a deployed standalone software application. In some examples, requesting service 102 is, or includes, an integration, widget, etc. Nevertheless, requesting service 102 transmits a request across network 104 (e.g., the Internet, etc.) to a consent processing and records retrieval platform 112 to request information related to a data owner.

In response, consent processing and records retrieval platform 112 acquires consent from the data owner and information corresponding to the received request from a record data silo 132. Record data silo 132 may be a database, decentralized repository, cloud storage, etc. solution for storing information such as, for example and without imputing limitation, medical records, personal information, health data information, financial information, and various other types of protected and/or sensitive data. Based on the request and the consent provided by the data owner via a patient device 106, consent processing and records retrieval platform 112 then transmits the acquired information to requesting service 102. In general, patient device 106 can include, for example and without imputing limitation, mobile devices, smartphones, personal computers, laptops, and various other computing systems. Additionally, while a single record data silo 132 is depicted as part of operating environment 100, it is understood that consent processing and records retrieval platform 112 may retrieve information from a plurality of different data stores, which may each utilize distinct APIs, protocols, and the like.

Requesting service 102 includes a record store 108 and a record querier 110. Requesting service 102 translates, or receives directly, based on integration with data requester systems, the information request which is then provided to record querier 110. Record querier 110 transmits the information request to a request receiver process 114 of consent processing and records retrieval platform 112. When record querier 110 receives back corresponding health information (e.g., medical record, personal or protected information, etc.) responsive to the transmitted information request, the corresponding health information is then provided to record store 108 for storage and later use and review by the data requester. In some examples, record store 108 may be an encrypted, privileged, or otherwise secured data repository.

Consent processing and records retrieval platform 112 includes request receiver 114 and various other processes and services for managing information requests and data owner consent. In some examples, consent processing and records retrieval platform 112 may architected as a monolith application. In some examples, consent processing and records retrieval platform 112 may be architected as a disaggregated platform such as, for example and without imputing limitation, a service mesh, a micro-services network, etc.

In particular, consent processing and records retrieval platform 112 includes a records retrieval process 116 which receives the information request from request receiver 114. Record retrieval process 116 manages retrieving the requested information and corresponding consent to distribute the requested information. Record retrieval process 116 identifies the data owner based on the information request and interfaces with a consent retrieval service 118 to retrieve consent from the data owner. Consent retrieval process 118 then retrieves consent from the data owner via patient device 106 or a stored consent profile imputing consent to certain types of requests (e.g., based on content requested, based on data requester, based on prior consents and/or a timing window, etc.).

In some examples, consent processing and records retrieval platform 112 includes a consent profiles store 136. Consent profiles store 136 stores consent profiles associated with individual data owners. In some examples, consent profiles are predetermined by the respective data owner ahead of time (e.g., through an account settings, preferences, etc.). In some examples, consent profiles may be automatically updated and/or modified based on downstream or parallel stream processing or the like.

Consent retrieval process 118 provides retrieved consent to record retrieval process 116 to verify that retrieved health information records may be provided to requesting service 102. In some examples, record retrieval process 116 retrieves the requested health information independent of consent and transmits the retrieved information only upon receipt and/or verification of data owner consent. In some examples, record retrieval process 116 may include the retrieved consent as part of a health information request provided to record data silo 132 for information retrieval.

Nevertheless, record retrieval process 116 identifies one or more appropriate record data silos 132 from which to retrieve the requested information based on the information request and/or the consent. Record data silos 132 may be associated with corresponding SILO APIs 120, over which consent processing and records retrieval platform 112 can retrieve requested information.

Patient device 106 may include a consent application 122 for retrieving data owner consent to information requests. Consent application 122 includes a request receiver 126, which receives consent requests from consent processing and records retrieval platform 112 and provides the receive consent requests to the data owner (e.g., via SMS text, alert, popup, email, application notification, etc.). Consent application 122 additionally includes a consent transmit process 128 for relaying data owner consent back to consent processing and records retrieval platform 112.

Further, consent application 122 may include a consent process and validator 124. Consent process and validator 124 receives and, in some examples, validates consent from the data owner. For example, consent may be associated with a password, biometric authentication (e.g., retina, face, thumb print, etc.), or the like. In some examples, consent process and validator 124 may include hardware keys, encryption controls (e.g., public/private keys, etc.) and the like. In effect, consent process and validator 124 ensures that the data owner is consenting to the information request rather than an impostor. Once the data owner consent has been validated, it is provided back to consent processing and records retrieval platform 112 via consent transmit 128 and the requested information is retrieved and/or provided to requesting service 102.

Further, a logging service 134 may overlay consent processing and records retrieval platform 112. Logging service 134 processes and monitors requests and internal transmissions in order to generate a detailed log of requests, retrievals, accesses, consents, and the like related to information requests received by consent processing and records retrieval platform 112. In some examples, both successful and failed information requests (e.g., due to lack of consent, incorrect data owner information, lack of authorization, etc.) are logged by logging service 134 and stored for later retrieval and review.

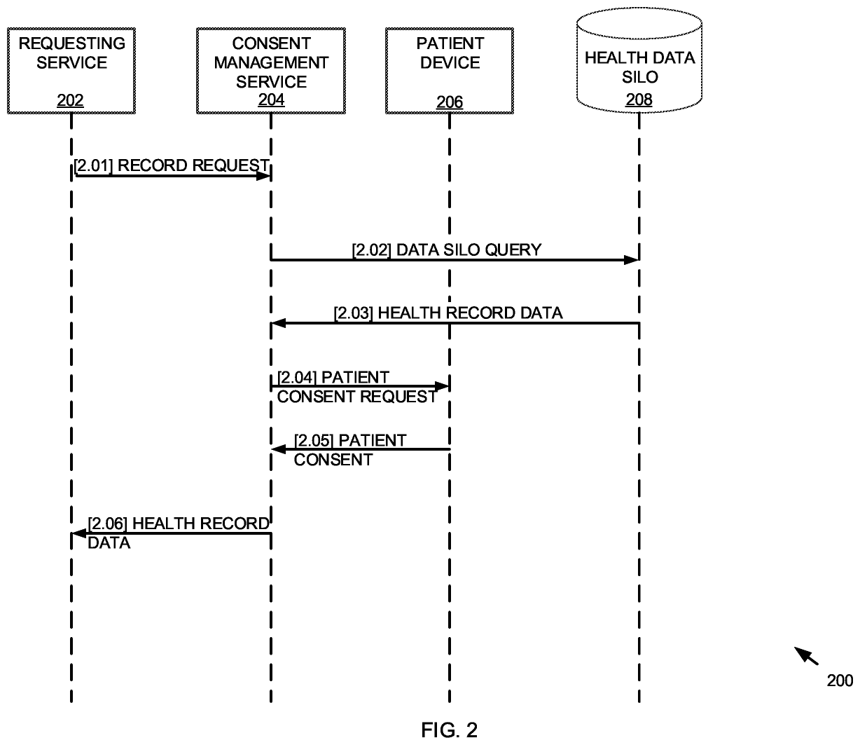

FIG. 2 is a sequence diagram 200 depicting a messaging sequence between services for managing consent and information requests. In particular, messages are passed between a requesting service 202, a consent management service 204, a patient device 206, and a health data silo 208 to provide health information to requesting service 202 retrieved from health data silo 208 and consented to by a respective owner via patient device 206. While health data and patients are referred to in sequence diagram 200, it is understood that such references are for explanatory purposes only and not to be understood as limiting. Various other types of data and data owners may consent to information requests without departing from the spirit and scope of the disclosure.

Requesting service 202 transmits to consent management service 204 a message 2.01 requesting a record. In some examples, requesting service 202 may include a translation layer, interface, or integration for adapting a third-party system query into a format and/or protocol suitable for consent management service 204. In some examples, record request message 2.01 includes data owner information such as, for example and without imputing limitation, name, address, zip code, unique identifier (e.g., social security number, insurance identifier, etc.), phone number, and the like.

Consent management service 204 transmits to health data silo 208 a message 2.02 including a data silo query. Consent management service 204 uses content of the received message 2.01 requesting a record to determine an appropriate health data silo 208 from which to retrieve the requested information. In some examples, an appropriate API, protocol, or interface corresponds to health data silo 208 and consent management service 204 is configured to generate a compliant request to health data silo 208 based on message 2.01.

Health data silo 208 transmits back to consent management service 204 a message 2.03 including health record data. The health record data may be in various formats and file types. In general, consent management service 204 is configured to receive message 2.03 and appropriately process and parse the included health record data. In some examples, health data silo 208 may fail to find any associated health record data. In such a case, a null, or empty, result may be returned to consent management service 204 as part of message 2.03. Nevertheless, consent management service 204 then transmits to patient device 206 a message 2.04 requesting patient consent.

In response, patient device 206 transmits to consent management service 204 a message 2.05 including patient consent. In some examples, message 2.05 includes additional information, such as restraints and/or rules associated with the patient consent. For example, and without imputing limitation, the patient may consent to only records of the last five years being provided to requesting service 202, or the patient may consent to only specified types of health record data (e.g., just medication information, just treatment information, etc.) being provided or the like. As a result, said consent information may additionally be included in message 2.05, or in an earlier configuration process, for consent management service 204 to process the health record data accordingly.

Consent management service 204 transmits to requesting service 202 a message 2.06 including the health record data. In some examples, consent management service 204 is configured to further process the health record data prior to providing a modified copy to requesting service 202. For example, where consent includes additional constraints and/or rules, consent management service 204 may cause the retrieved health records to be appropriately modified in accordance with the constraints and/or rules, such as by redacting information, removing files and/or data from the health data record transmitted to consent management service 204, etc. Further, in some examples, consent management service 204 removes the retrieved health data record from any internal systems once the health data record has been provided to requesting service 202 or within some predetermined amount of time thereafter. In some examples, consent management service 204 includes, or interfaces with, a logging service or the like in order to maintain an audit trail of all requests and processes included in sequence diagram 200.

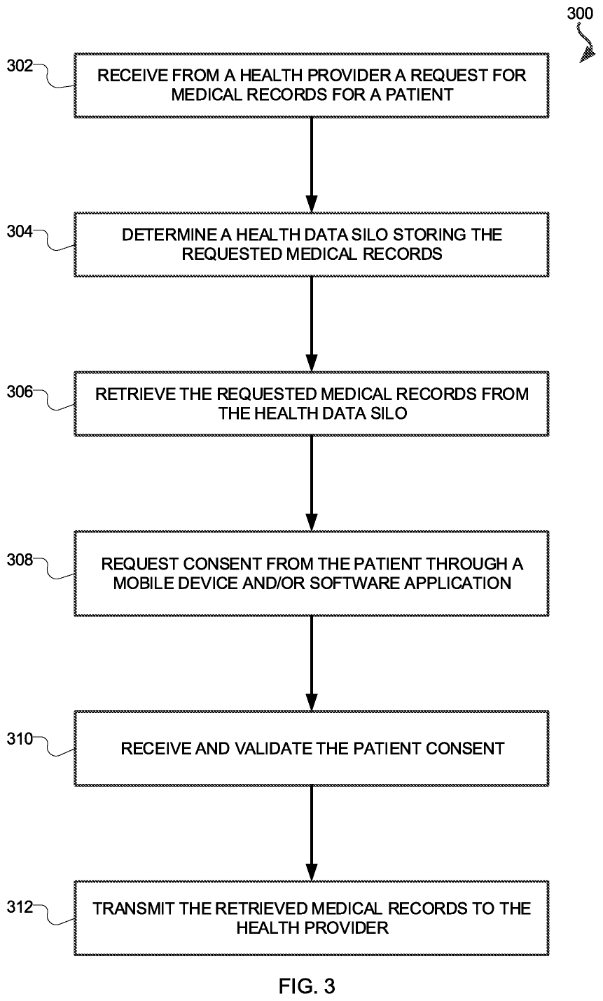

FIG. 3 depicts a method 300 for managing consent and medical records retrieval. While medical records for a patient are disclosed in method 300, it is understood that they are for explanatory purposes only and not to be taken to limit the disclosure to solely patient medical records. Rather, as discussed above, method 300 may be applied to requests for, and distribution of, information based on consent of a corresponding data owner (e.g., patient, person associated with personally identifying information (PII), client, etc.).

At step 302, a request is received from a health provider for medical records for a patient. For example, a health provider may be onboarding a new patient and submit a medical records request to determine the current health status and history of the new patient.

At step 304, it is determined which health data silo may store the requested information. In some examples, the request itself may include identification of appropriate health data silos. In some examples, a database or lookup table of appropriate health data silos may be consulted based on the received request.

At step 306, the requested medical records are retrieved from the health data silo. An additional query or request may be provided to the determined health data silo based on the received request, such as where the health data silo is interfaced with over a particular protocol or API.

At step 308, consent is requested from the patient through a mobile device and/or software application. For example, an application installed on the patient's smartphone may alert the patient of a consent request for disbursement of medical records. In some examples, the alert may include information about the requesting party (e.g., time of request, requesting physician or clinic, etc.). The patient may then consent to the request through the application.

At step 310, the patient consent is received and validated. Validation information may be included in a transmission from the patient mobile device and/or matched against stored records or the like. For example, biometric information received through the mobile device, a password, a personal identification number (PIN), encryption keys, or the like may be used to validate the consent as having come from the intended patient.

At step 312, having received validated consent, the retrieved medical records are transmitted to the health provider. In some examples, further processing based on the consent may be applied to the retrieved medical records before modified versions of the retrieved medical records are transmitted to the health provider. Further, in some examples, a record of the entire transaction may be logged and stored. Additionally, the patient may receive a copy of the record or the like. As a result, the health provider is able to request and receive consented to medical records through a seamless process without requiring duplicative or voluminous paperwork from either the patient or the health provider, while maintaining regulation compliant audit trails and the like of the transaction.

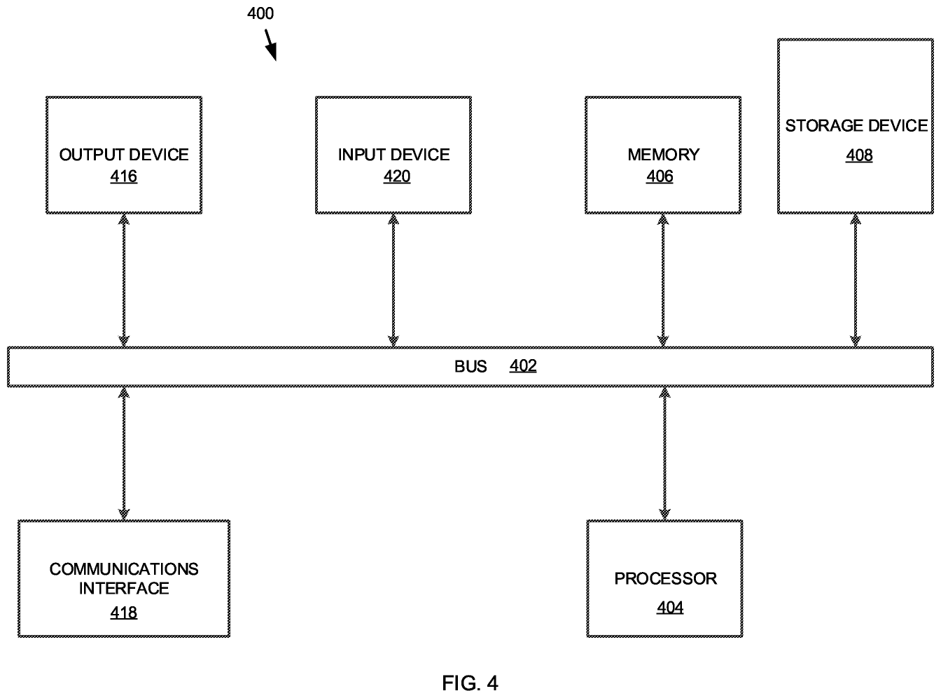

FIG. 4 is an example computing system 400 that may implement various systems and methods discussed herein. The computer system 400 includes one or more computing components in communication via a bus 402. In one implementation, the computing system 400 includes one or more processors 404. The processor 404 can include one or more internal levels of cache (not depicted) and a bus controller or bus interface unit to direct interaction with the bus 402. The processor 404 can perform calculations on data, and specifically implements the various processes and systems discussed herein. Main memory 406 may include one or more memory cards and a control circuit (not depicted), or other forms of removable memory, and may store various consent management applications including computer executable instructions, that when run on the processor 404, implement the methods and systems set out herein. Other forms of memory, such as a storage device 408, may also be included and accessible, by the processor (or processors) 404 via the bus 402.

The computer system 400 can further include a communications interface 418 by way of which the computer system 400 can connect to networks and receive data useful in executing the methods and system set out herein as well as transmitting information to other devices. The computer system 400 can include an output device 416 by which information is displayed, such as a display (not depicted). The computer system 400 can also include an input device 420 by which information is input. Input device 420 can also be a scanner, keyboard, and/or other input devices for human interfacing as will be apparent to a person of ordinary skill in the art. The system set forth in FIG. 4 is but one possible example of a computer system that may employ or be configured in accordance with embodiments of the present disclosure. It will be appreciated that other non-transitory tangible computer-readable storage media storing computer-executable instructions for implementing the presently disclosed technology on a computing system may be utilized.

In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the methods can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a computer-readable storage medium having stored thereon instructions, which may be used to program a computer system (or other electronic devices) to perform a process according to the present disclosure. A computer-readable storage medium includes any mechanism for storing information in a form (e.g., software, processing application) readable by a computer. The computer-readable storage medium may include, but is not limited to, optical storage medium (e.g., CD-ROM), magneto-optical storage medium, read only memory (ROM), random access memory (RAM), erasable programmable memory (e.g., EPROM and EEPROM), flash memory, or other types of medium suitable for storing electronic instructions.

The description above includes example systems, methods, techniques, instruction sequences, and/or computer program products that embody techniques of the present disclosure. However, it is understood that the described disclosure may be practiced without these specific details.

While the present disclosure has been described with references to various implementations, it will be understood that these implementations are illustrative and that the scope of the disclosure is not limited to them. Many variations, modifications, additions, and improvements are possible. More generally, implementations in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. A computer-implemented method for delivering consented to information, the method comprising:
receiving, from a data requester, a request for consented to information associated with a data owner;
retrieving, from the data owner, consent to deliver the consented to information to the data requester;
determining, based on the request and the data owner, a data store from which to retrieve the consented to information;
retrieving, from the determined data store, the consented to information; and
delivering the consented to information to the data requester.

2. The method of claim 1, further comprising validating the retrieved consent, wherein validation is based on one or more of biometric information, a password, a code, or a key.

3. The method of claim 1, further comprising modifying the retrieved consented to information based on the retrieved consent, and wherein the modified retrieved consented to information is delivered to the data requester.

4. The method of claim 1, further comprising generating a consent profile for the data owner, and wherein retrieving the consent from the data owner includes accessing the generated consent profile.

5. The method of claim 1, wherein the consent is retrieved from the data owner through a mobile application deployed to a mobile device.

6. The method of claim 1, wherein retrieving the consent to information from the determined data store further comprises generating a second request based on the received request, the second request complying with one of an application programming interface (API) or a protocol corresponding to the determined data store.

7. The method of claim 1, wherein one or more of the data requester is a health provider, the data owner is a patient, or the requested consented to information associated with the data owner is part of a medical record.

8. A system for delivering consented to information, the system comprising:
one or more processors; and
a memory comprising instructions to:
receive, from a data requester, a request for consented to information associated with a data owner;
retrieve, from the data owner, consent to deliver the consented to information to the data requester;
determine, based on the request and the data owner, a data store from which to retrieve the consented to information;
retrieve, from the determined data store, the consented to information; and
deliver the consented to information to the data requester.

9. The system of claim 8, wherein the memory further comprises instructions to validate the retrieved consent, wherein validation is based on one or more of biometric information, a password, a code, or a key.

10. The system of claim 8, wherein the memory further comprises instructions to modify the retrieved consented to information based on the retrieved consent, and wherein the modified retrieved consented to information is delivered to the data requester.

11. The system of claim 8, wherein the memory further comprises instructions to generate a consent profile for the data owner, and wherein retrieving the consent from the data owner includes accessing the generated consent profile.

12. The system of claim 8, wherein the consent is retrieved from the data owner through a mobile application deployed to a mobile device.

13. The system of claim 8, wherein retrieving the consent to information from the determined data store further comprises generating a second request based on the received request, the second request complying with one of an application programming interface (API) or a protocol corresponding to the determined data store.

14. The system of claim 8, wherein one or more of the data requester is a health provider, the data owner is a patient, or the requested consented to information associated with the data owner is part of a medical record.

15. A non-transitory computer readable medium storing instructions that, when executed by one or more processors, cause the one or more processors to:
receive, from a data requester, a request for consented to information associated with a data owner;
retrieve, from the data owner, consent to deliver the consented to information to the data requester;
determine, based on the request and the data owner, a data store from which to retrieve the consented to information;
retrieve, from the determined data store, the consented to information; and
deliver the consented to information to the data requester.

16. The non-transitory computer readable medium of claim 15, further storing instructions to validate the retrieved consent, wherein validation is based on one or more of biometric information, a password, a code, or a key.

17. The non-transitory computer readable medium of claim 15, further storing instructions to modify the retrieved consented to information based on the retrieved consent, and wherein the modified retrieved consented to information is delivered to the data requester.

18. The non-transitory computer readable medium of claim 15, further storing instructions to generate a consent profile for the data owner, and wherein retrieving the consent from the data owner includes accessing the generated consent profile.

19. The non-transitory computer readable medium of claim 15, wherein the consent is retrieved from the data owner through a mobile application deployed to a mobile device.

20. The non-transitory computer readable medium of claim 15, wherein retrieving the consent to information from the determined data store further comprises generating a second request based on the received request, the second request complying with one of an application programming interface (API) or a protocol corresponding to the determined data store.
